(19) United States
(12) Patent Application Publication
(10) Pub. No.: US 2020/0155037 A1
(43) Pub. Date: May 21, 2020

(54) STORAGE MEDIUM, INFORMATION PROVIDING METHOD, AND INFORMATION PROVIDING DEVICE

(71) Applicant: HONDA MOTOR CO., LTD., Tokyo (JP)

(72) Inventors: Yasushi Ikeuchi, Wako-shi (JP); Takeyuki Suzuki, Wako-shi (JP); Naoko Imai, Wako-shi (JP)

(21) Appl. No.: 16/687,846

(22) Filed: Nov. 19, 2019

(30) Foreign Application Priority Data
Nov. 21, 2018 (JP) 2018-218357

(51) Int. Cl. A61B 5/11; G06F 3/01; G06N 20/00; G16H 10/20

(52) U.S. Cl. CPC A61B 5/112; G06F 3/011; A61B 2562/0219; G06N 20/00; G16H 10/20; G06F 3/017

(57) ABSTRACT

A computer-readable non-transitory storage medium is provided storing a program causing a computer to acquire physical information of a user, and determine whether or not it is effective to apply an assistance device assisting in a personal motion to the user on the basis of the acquired physical information.

### CROSS-REFERENCE TO RELATED APPLICATION

Priority is claimed on Japanese Patent Application No. 2018-218357, filed Nov. 21, 2018, the content of which is incorporated herein by reference.

### BACKGROUND OF THE INVENTION

### Field of the Invention

The present invention relates to a storage medium, an information providing method, and an information providing device.

### Description of Related Art

In the related art, a medical information providing system that provides medical information to a user is known. In this medical information providing system, for example, a scheme for preparing a database in which medical information such as treatment methods, advice, and medical institutions has been stored, extracting the medical information from the database using information input by the user as search conditions, and providing the medical information to the user has been adopted (for example, Japanese Unexamined Patent Application, First Publication No. 2007-172275).

In a scheme of the related art, it is possible to provide medical information such as a treatment method suitable for a user, but it is not possible to provide information on an assistance device assisting in various motions of the user. Also when a doctor or the like examines patients in a medical institution, there is a lack of knowledge indicating whether an assistance device may be used for patients with certain symptoms at a certain timing, and this is a factor that hinders spreading of assistance devices.

### SUMMARY OF THE INVENTION

The present invention has been made in view of such circumstances, and an object of the present invention is to provide a storage medium, an information providing method, and an information providing device that can provide information on an assistance device suitable for a user.

A storage medium, an information providing method, and an information providing device according to the present invention adopt the following configurations.

(1) A computer-readable non-transitory storage medium storing a program according to an aspect of the present invention, the program causing a computer to: acquire physical information of a user; and determine whether or not it is effective to apply an assistance device assisting in a personal motion to the user on the basis of the acquired physical information.

(2) In the aspect (1), the program is configured to cause the computer to determine whether or not it is effective to apply the assistance device to the user in a case where the physical information includes information on skeletal movement trouble of the user.

(3) In the above aspect (1), the assistance device is a walking assistance device, and the program is configured to cause the computer to determine that it is effective to apply the assistance device to the user in a case where there is periodicity in a walking motion of the user indicated by the physical information.

(4) In the above aspect (1), the assistance device is a walking assistance device, and the program is configured to cause the computer to determine that it is effective to apply the assistance device to the user in a case where bilateral symmetry of a walking motion of the user indicated by the physical information deteriorates.

(5) In the aspect (1), the program is configured to cause the computer to determine a type of assistance device suitable for the user on the basis of the acquired physical information in a case where it has been determined that it is effective to apply the assistance device to the user.

(6) In the above aspect (1), the program is configured to cause the computer to further acquire a result which is an answer of the user to a question about the physical information, and determine whether or not it is effective to apply the assistance device to the user on the basis of both the acquired answer result and the acquired physical information.

(7) In the above aspect (1), the program is configured to cause the computer to generate a page on which an answer to a question about the physical information is able to be input and a page on which measurement data regarding a body is able to be acquired.

(8) In the above aspect (1), the program is configured to cause the computer to determine whether or not it is effective to apply the assistance device to the user, using a model learned to output application information for the assistance device when the physical information is input.

(9) An information providing method according to an aspect of the present invention is an information providing method including: acquiring, by a computer, physical information of a user; and determining, by the computer, whether or not it is effective to apply an assistance device assisting in a personal motion to the user on the basis of the acquired physical information.

(10) An information providing device according to an aspect of the present invention is an information providing device including: an acquirer configured to acquire physical information of a user; and a first determiner configured to determine whether or not it is effective to apply an assistance device assisting in a personal motion to the user on the basis of the physical information acquired by the acquirer.

According to (1) to (4), (9), and (10), it is possible to provide information on the assistance device suitable for the user by acquiring the physical information of the user and determining whether or not it is effective to apply an assistance device assisting in a personal motion to the user on the basis of the acquired physical information.

According to (5), it is possible to provide information on the type of assistance device suitable for the user by determining the type of assistance device suitable for the user on the basis of the acquired physical information in a case where it has been determined that it is effective to apply the assistance device to the user.

According to (6) and (7), it is possible to provide information on the assistance device more suitable for the user in consideration of both answer information and the physical information of the user by further acquiring the result which is an answer of the user to the question about the physical information and determining whether or not it is effective to apply the assistance device to the user on the basis of both the acquired answer result and the acquired physical information.

According to (8), it is possible to provide information on the assistance device for a variety of pieces of physical information by determining whether or not it is effective to apply the assistance device to the user, using a model learned to output application information of the assistance device when the physical information has been input.

### BRIEF DESCRIPTION OF THE DRAWINGS

FIG. 1 is a diagram showing an overview of a medical information providing system of a first embodiment.

FIG. 2 is a flowchart showing an example of a process of the medical information providing device of the first embodiment.

FIG. 3 is a diagram showing an overview of a medical information providing system of a second embodiment.

FIG. 4 is a sequence diagram showing an example of a process of the medical information providing device and a terminal device according to the second embodiment.

FIG. 5 is a diagram showing an example of a page that is displayed on the terminal device of the second embodiment.

FIG. 6 is a functional block diagram of a medical information providing device of a third embodiment.

FIG. 7 is a diagram showing a configuration of a determination model according to the third embodiment.

FIG. 8 is a flowchart showing an example of a process of the medical information providing device of the third embodiment.

### DETAILED DESCRIPTION OF THE INVENTION

Hereinafter, embodiments of a storage medium, an information providing method, and an information providing device according to the present invention will be described with reference to the drawings.

### First Embodiment

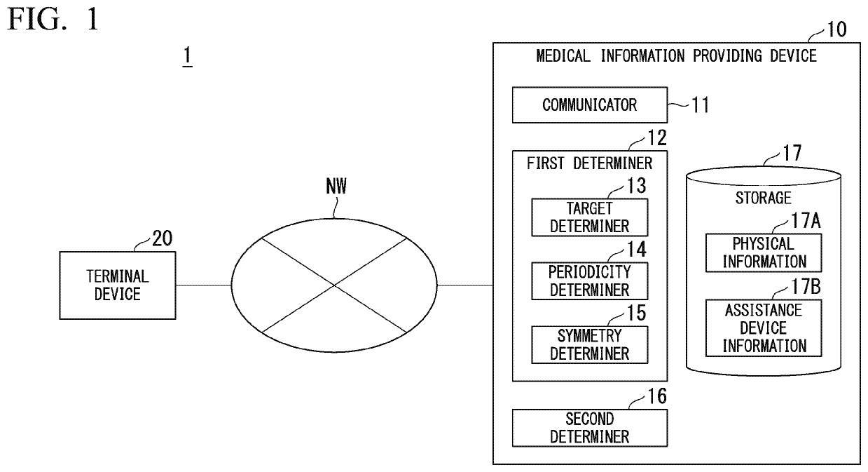

FIG. 1 is a diagram showing an overview of a medical information providing system 1 according to a first embodiment. The medical information providing system 1 is a system that determines whether or not it is effective to apply an assistance device that assists in a personal motion to a target on the basis of physical information of the target, and provides a determination result. Examples of the assistance device include a device that assists in a motion of a person having a motion disability, such as a walking assistance device that assists in the walking of a person, and an assistance device that assists in movement of the arms of the person. Hereinafter, a case in which the assistance device is the walking assistance device will be described as an example.

The medical information providing system 1 includes, for example, a medical information providing device 10 (an information providing device) and a terminal device 20. The medical information providing device 10 and the terminal device 20 are connected to each other via a network NW, and communicate with each other via the network NW. The network NW is a wired or wireless network. Examples of the network NW include a wide area network (WAN), a local area network (LAN), the Internet, and a dedicated line.

### [Configuration of Medical Information Providing Device 10]

The medical information providing device 10 determines whether or not it is effective to apply the assistance device to the target on the basis of the physical information of the target, and outputs a determination result. The physical information of the target includes information on a walking motion of the target. Examples of the information on the walking motion of the target include acceleration data and angular velocity data of the target at the time of walking.

The medical information providing device 10 includes, for example, a communicator 11 (an acquirer), a first determiner 12, a second determiner 16, and a storage 17. Each of these components is realized, for example, by a hardware processor such as a central processing unit (CPU) executing a program (software). Some or all of the components may be realized by hardware (including circuitry) such as a large scale integration (LSI), an application specific integrated circuit (ASIC), a field-programmable gate array (FPGA), or a graphics processing unit (GPU) or may be realized by software and hardware in cooperation. The program may be stored in a storage device (a storage device including a non-transitory storage medium) such as a hard disk drive (HDD) or a flash memory in advance or may be stored in a detachable storage medium (a non-transitory storage medium) such as a DVD or a CD-ROM and installed by the storage medium being mounted in the drive device.

The communicator 11 includes a communication interface for performing communication between devices via the network NW. For example, the communicator 11 receives the physical information of the target transmitted by the terminal device 20, and transmits a determination result of whether or not it is effective to apply the assistance device, to the terminal device 20. The communicator 11 stores the acquired physical information in the storage 17.

The first determiner 12 determines whether or not it is effective to apply the assistance device to the target on the basis of the physical information of the target acquired by the communicator 11. In a case where the physical information of the target acquired by the communicator 11 includes information on skeletal movement trouble of the target, the first determiner 12 determines whether or not it effective to apply the assistance device to the target. The first determiner 12 includes, for example, a target determiner 13, a periodicity determiner 14, and a symmetry determiner 15.

The target determiner 13 determines whether or not it is effective to apply the assistance device to the target on the basis of a determination result of the periodicity determiner 14, for example. The target determiner 13 determines an operation mode of the assistance device in a case where the assistance device is applied to the target on the basis of a determination result of the symmetry determiner 15. Examples of the operation mode include a symmetry improvement mode for improving bilateral symmetry at the time of walking, and a stride improvement mode for improving a stride at the time of walking.

The periodicity determiner 14 determines whether or not there is periodicity in a walking motion indicated by the physical information of the target. The periodicity in the walking motion indicates that there is periodicity in a repeated motion of alternately placing a left foot and a right foot forward. For example, the periodicity determiner 14 detects a landing timing of the foot of the target using the acceleration data and the angular velocity data. Then, the periodicity determiner 14 determines whether or not there is the periodicity on the basis of whether or not an average value of intervals between the detected landing timings is smaller than a predetermined threshold value. In a case where there is the periodicity in the walking motion, it is estimated that the target can perform a certain walking motion. On the other hand, for example, in a case where the walking motion is halting by one step at a time, it is determined that there is no periodicity. The target determiner 13 determines that it is effective to apply the assistance device to the target in a case where the periodicity determiner 14 has determined that there is the periodicity. On the other hand, the target determiner 13 determines that it is not effective to apply the assistance device to the target in a case where the periodicity determiner 14 has determined that there is no periodicity.

The symmetry determiner 15 determines whether or not the bilateral symmetry of the walking motion of the left foot and the right foot deteriorates in the walking motion indicated by the physical information of the target. For example, the symmetry determiner 15 detects the landing timing of the foot of the target using the acceleration data and the angular velocity data. Then, the symmetry determiner 15 determines whether the bilateral symmetry of the walking motion deteriorates on the basis of the interval between the detected landing timings. The walking assistance device can improve the symmetry in a case where the bilateral symmetry of the walking motion deteriorates. In a case where the symmetry determiner 15 has determined that the bilateral symmetry deteriorates, the target determiner 13 determines that it is effective to apply an assistance device operating in a mode for improving symmetry. On the other hand, in a case where the symmetry determiner 15 has determined that the bilateral symmetry does not deteriorate, the target determiner 13 determines that it is effective to apply the assistance device operating in an operation improvement mode (for example, the stride improvement mode) other than the mode for improving symmetry.

The second determiner 16 determines a type (condition) of the assistance device suitable for the target on the basis of the determination result of the first determiner 12. For example, the second determiner 16 determines the type of assistance device suitable for the target by referring to assistance device information stored in the storage 17. The assistance device information includes specification data of various assistance devices. For example, in a case where the first determiner 12 determines that it is effective to apply an assistance device operating in the mode for improving symmetry, the second determiner 16 determines that an assistance device excellent in improving the bilateral symmetry is a type of assistance device suitable for the target. Hereinafter, the determination result of the first determiner 12 and a determination result of the second determiner 16 are collectively referred to as “application information for the assistance device”.

The storage 17 stores, for example, physical information 17A of the target acquired by the communicator 11 and assistance device information 17B. The storage 17 includes, for example, a hard disk drive (HDD), a flash memory, an electrically erasable programmable read only memory (EEPROM), a read only memory (ROM), or a random access memory (RAM).

### [Configuration of Terminal Device 20]

The terminal device 20 is a device that is used by a user. Examples of the terminal device 20 include a mobile phone such as a smartphone, a tablet computer, a notebook computer, or a desktop computer. Examples of the user include targets who desire application information for an assistance device to be provided to themselves, medical personnel who desire application information for an assistance device for a target (patient) to be provided, and a person in charge of a company that provides assistance devices. The user operates the terminal device 20 to transmit the physical information of the target (information on a malfunction of a body) to the medical information providing device 10. The user can refer to the application information for the assistance device provided by the medical information providing device 10 and displayed on the display device of the terminal device 20.

[Processing Flow of Medical Information Providing Device 10]

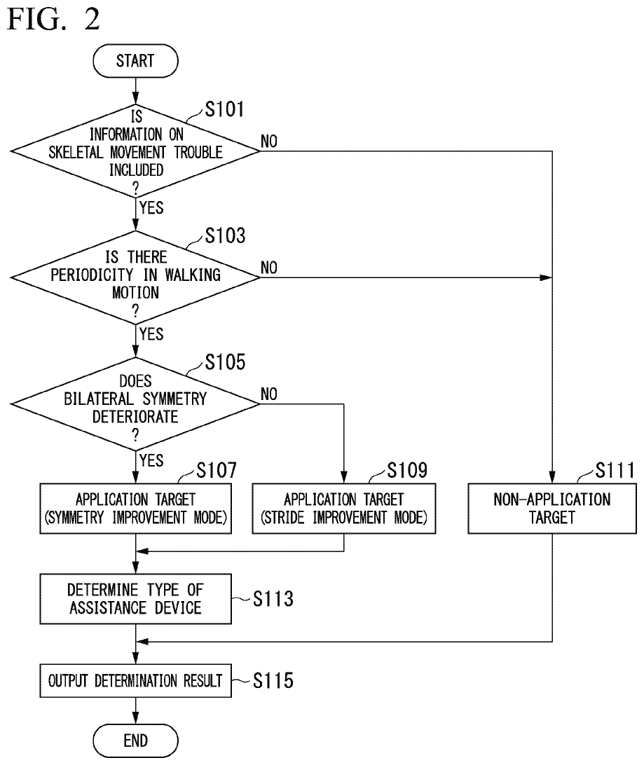

Next, a processing flow of the medical information providing device 10 will be described. FIG. 2 is a flowchart showing an example of a process of the medical information providing device 10 according to the first embodiment. The flowchart shown in FIG. 2 is started, for example, when the medical information providing device 10 has received the physical information of the target from the terminal device 20.

First, the target determiner 13 determines whether or not the physical information (symptoms, treatment, progress, or the like) of the target transmitted by the terminal device 20 includes information on the skeletal movement trouble (step S101). For example, in a case where the physical information includes information on a trouble with bending and stretching of joints, the target determiner 13 determines that the physical information includes the information on the skeletal movement trouble. In a case where the target determiner 13 determines that the physical information does not include the information on the skeletal movement trouble, the target determiner 13 determines that it is not effective to apply the assistance device to the target, that is, the target is a non-application target (step S111).

On the other hand, in a case where it is determined that the physical information of the target acquired by the target determiner 13 includes the information on the skeletal movement trouble, the periodicity determiner 14 determines whether or not there is periodicity in the walking motion indicated by the acquired physical information of the target (step S103). In a case where the periodicity determiner 14 has determined that there is no periodicity, the target determiner 13 determines that it is not effective to apply the assistance device to the target, that is, the target is a non-application target (step S111).

On the other hand, in a case where the periodicity determiner 14 has determined that there is periodicity, the symmetry determiner 15 determines whether or not the bilateral symmetry of the walking motion of the left foot and the right foot in the walking motion indicated by the physical information of the target deteriorates (step S105). In a case where the symmetry determiner 15 has determined that the bilateral symmetry of the walking motion deteriorates, the target determiner 13 determines that it is effective to apply an assistance device operating in the mode for improving symmetry (step S107). On the other hand, in a case where the symmetry determiner 15 has determined that the bilateral symmetry of the walking motion does not deteriorate, the target determiner 13 determines that it is effective to apply the assistance device operating in the operation improvement mode (for example, the stride improvement mode) other than the mode for improving the symmetry (step S109).

After an operation mode of the assistance device has been determined by the target determiner 13, the second determiner 16 determines a type of assistance device suitable for the target (step S113). For example, the medical information providing device 10 determines a type of assistance device suitable for the target by referring to the assistance device information 17B stored in the storage 17.

Then, the target determiner 13 outputs the application information for the assistance device to the terminal device 20 via the communicator 11 (step S115). Thus, the process of the flowchart ends.

According to the first embodiment described above, it is possible to provide information on the assistance device suitable for the target. The example in which the determination is made as to whether or not the assistance device can be applied to the target on the basis of the periodicity of the walking motion and the bilateral symmetry of the walking motion has been described in the first embodiment, but the present invention is not limited thereto. The medical information providing device 10 may determine whether or not the assistance device can be applied to the target on the basis of any feature information on the walking motion.

In a case where the second determiner 16 has determined that there is no assistance device suitable for the target, information on conditions for an assistance device which would be determined to be effective may be provided to the user (for example, a person in charge of the company that provides the assistance device). By collecting such information on the condition of the assistance device, it is possible to accumulate knowledge about an assistance device to be newly developed.

### Second Embodiment

Hereinafter, a second embodiment will be described. A medical information providing system 1A of the second embodiment is different from that of the first embodiment in that the medical information providing device 10A transmits a page on which an answer to a question about a body can be input and a page on which measurement data regarding the body can be acquired, to a terminal device 20A, and determines whether or not an assistance device can be applied on the basis of both a result of the answer to the question and physical information obtained from these pages. Therefore, for a configuration or the like, the drawings and related description described in the first embodiment are used, and detailed description thereof will be omitted.

### [Configuration of Medical Information Providing Device 10A]

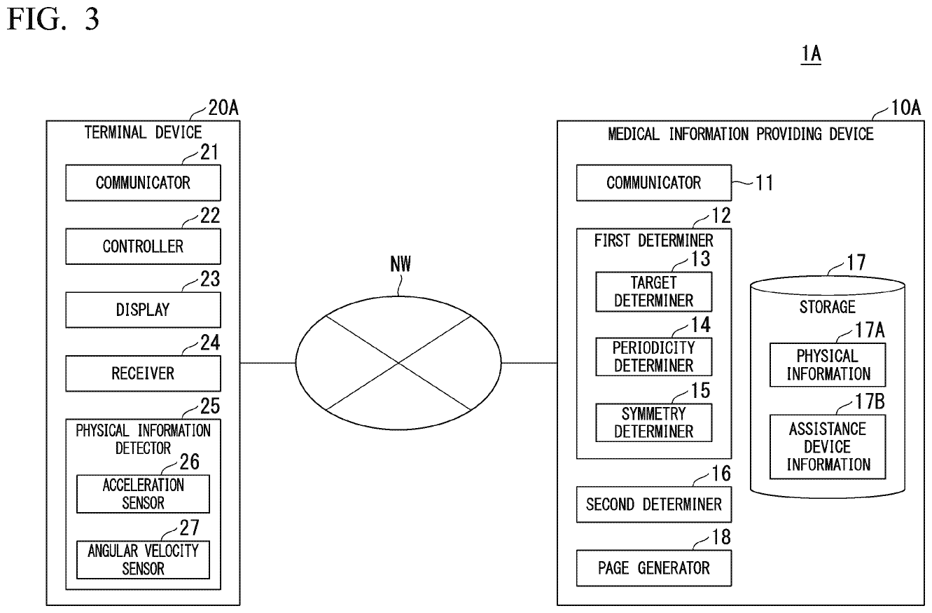

FIG. 3 is a diagram showing an overview of the medical information providing system 1A of the second embodiment. The medical information providing device 10A further includes, for example, a page generator 18, in addition to the communicator 11, the first determiner 12, the second determiner 16, and the storage 17 in the first embodiment. The page generator 18 generates, for example, a page that receives an input of an answer to a question about a body, and a page that enables measurement data regarding the body to be acquired. The page generator 18 transmits information on the generated page to the terminal device 20A via the communicator 11.

### [Configuration of Terminal Device 20A]

The terminal device 20A is, for example, a portable device such as a mobile phone such as a smartphone, a tablet computer, or a notebook computer. The terminal device 20A includes, for example, a communicator 21, a controller 22, a display 23, a receiver 24, and a physical information detector 25. The components of the terminal device 20A are realized, for example, by a hardware processor such as a CPU executing a program (software).

The communicator 21 includes a communication interface for performing communication between devices via a network NW. The controller 22 controls an entire operation of the terminal device 20A. The controller 22, for example, causes various pages received from the medical information providing device 10A to be displayed on the display 23, or causes physical information acquired by the physical information detector 25 to be transmitted to the medical information providing device 10A via the communicator 21.

The display 23 displays, for example, various pages received from the medical information providing device 10A. For example, the display 23 is, for example, a liquid crystal display (LCD) or an organic electroluminescence (EL) display device. The receiver 24 receives various operations of the user. For example, the receiver 24 is a touch panel, operation buttons, a keyboard, or the like. In a case where the display 23 is a touch panel compatible display, the display 23 may have a function of the receiver 24.

The physical information detector 25 detects the physical information of the user (the target) who operates the terminal device 20A. The physical information detector 25 includes, for example, an acceleration sensor 26 and an angular velocity sensor (a gyro sensor) 27. The acceleration sensor 26 detects an acceleration when the terminal device 20A moves, that is, an acceleration of the user carrying the terminal device 20A. The angular velocity sensor 27 detects an angular velocity when the terminal device 20A moves, that is, an angular velocity of the user carrying the terminal device 20A.

### [Processing Flow of Medical Information Providing Device 10A and Terminal Device 20A]

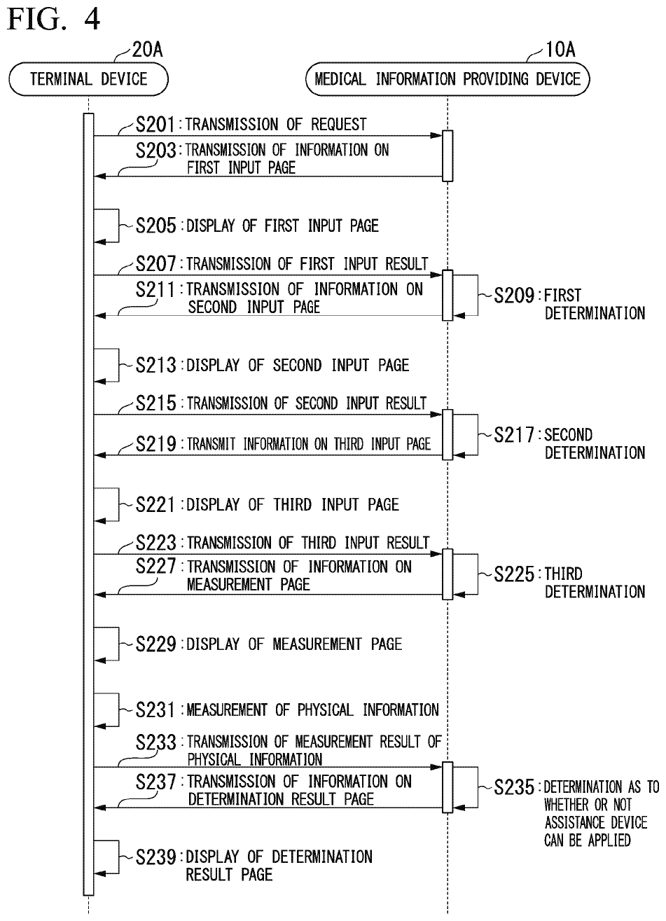

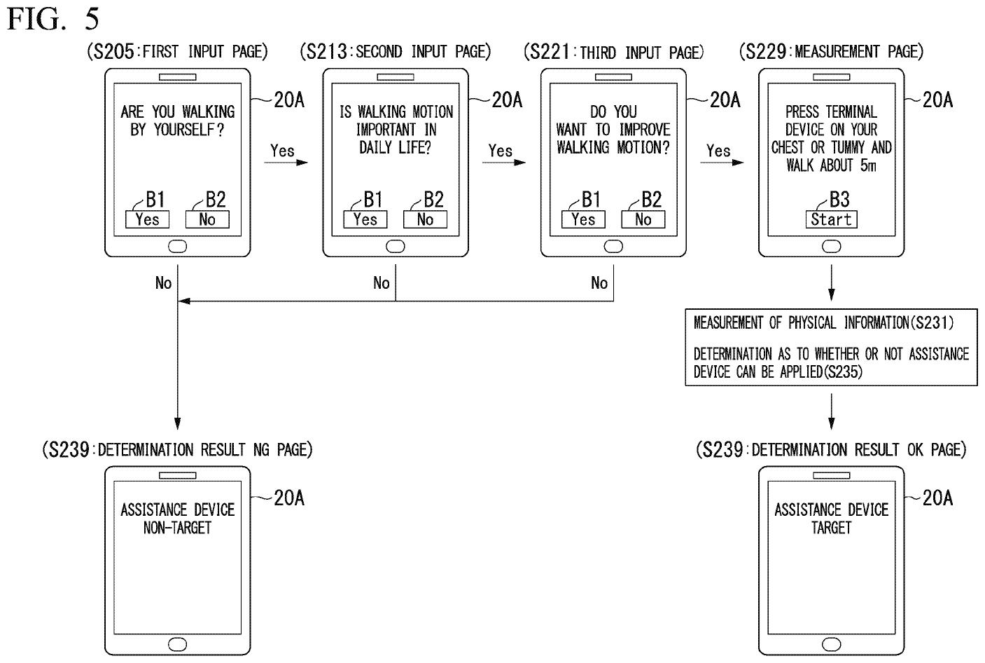

Next, a processing flow of the medical information providing device 10A and the terminal device 20A will be described. FIG. 4 is a sequence diagram showing an example of a process of the medical information providing device 10A and the terminal device 20A according to the second embodiment. FIG. 5 is a diagram showing an example of a page that is displayed on the terminal device 20A of the second embodiment.

First, the communicator 21 of the terminal device 20A transmits a request for a page that provides application information of the assistance device to the medical information providing device 10A on the basis of an operation of a user (step S201). In response to the request received from the terminal device 20A, the page generator 18 of the medical information providing device 10A generates a first input page that receives an input of an answer to a question about a body, and transmits the first input page to the terminal device 20A via the communicator 11 (step S203). The first input page is, for example, a page that receives an input of an answer about whether or not the user can walk by himself/herself.

Then, the display 23 of the terminal device 20A displays the first input page on the basis of information on the first input page received from the medical information providing device 10A (step S205). “S205: First input page” in FIG. 5 is an example of the first input page. The first input page includes a button B1 (Yes) and a button B2 (No) for receiving an answer to a question “Are you walking by yourself?”. The communicator 21 of the terminal device 20A transmits a first input result that is an answer to the above question to the medical information providing device 10A on the basis of the user pressing the button (step S207). The user walking by him/herself may include a case in which the user can walk alone him/herself using an assistive device such as a cane, in addition to a case in which the user can walk without using any assistance from another person or an assistive device of another person.

Then, the first determiner 12 of the medical information providing device 10A determines whether or not it is effective to apply the assistance device to the user on the basis of the first input result received from the terminal device 20A (a first determination) (step S209). In a case where the first input result is an answer indicating that the user does not walk by himself/herself (when the button B2 (No) has been pressed), the first determiner 12 causes a determination result NG page indicating that it is not effective to apply the assistance device to the user to be displayed on the terminal device 20A (step S239). “S239: Determination result NG page” in FIG. 5 is an example of the determination result NG page. The user can confirm that it is not effective to apply the assistance device by referring to the determination result NG page displayed on the display 23 of the terminal device 20A.

On the other hand, in a case where the first input result is an answer indicating that the user is walking by himself/herself (when the button B1 (Yes) has been pressed), the first determiner 12 of the medical information providing device 10A transmits information on a second input page that receives an input of the answer to the question about the body, to the terminal device 20A (step S211). The second input page is, for example, a page that receives an input of an answer about an importance of the walking motion of the user.

Then, the display 23 of the terminal device 20A displays the second input page on the basis of information on the second input page received from the medical information providing device 10A (step S213). “S213: Second input page” in FIG. 5 is an example of the second input page. The second input page includes a button B1 (Yes) and a button B2 (No) for receiving an answer to a question “Is walking motion important in daily life?”. The communicator 21 of the terminal device 20A transmits a second input result that is an answer to the above question to the medical information providing device 10A on the basis of the user pressing the button (step S215).

Then, the first determiner 12 of the medical information providing device 10A determines whether or not it is effective to apply the assistance device to the user on the basis of the second input result received from the terminal device 20A (a second determination) (step S217). In a case where the second input result is an answer indicating that the walking motion is not important (when the button B2 (No) has been pressed), the first determiner 12 of the medical information providing device 10A causes the determination result NG page indicating that it is not effective to apply the assistance device to the user to be displayed on the terminal device 20A (step S239). “S239: Determination result NG page” in FIG. 5 is an example of the determination result NG page. The user can confirm that it is not effective to apply the assistance device by referring to the determination result NG page displayed on the display 23 of the terminal device 20A.

On the other hand, in a case where the second input result is an answer indicating that the walking motion is important (when the button B1 (Yes) has been pressed), the first determiner 12 of the medical information providing device 10A transmits information on a third input page that receives an input of an answer to a question about a body to the terminal device 20A via the communicator 11 (step S219). The third input page is, for example, a page that receives an input of an answer about a demand for improvement of the walking motion of the user (a demand for walking reconstruction).

Then, the display 23 of the terminal device 20A displays the third input page on the basis of information on the third input page received from the medical information providing device 10A (step S221). “S221: Third input page” in FIG. 5 is an example of the third input page. The third input page includes a button B1 (Yes) and a button B2 (No) for receiving an answer to a question “Do you want to improve walking motion?”. The communicator 21 of the terminal device 20A transmits a third input result that is an answer to the above question to the medical information providing device 10A on the basis of the user pressing the button (step S223).

Then, the first determiner 12 of the medical information providing device 10A determines whether or not it is effective to apply the assistance device to the user on the basis of the third input result received from the terminal device 20A (a third determination) (step S225). In a case where the third input result is an answer indicating that improvement in walking motion is not desired (when the button B2 (No) has been pressed), the first determiner 12 of the medical information providing device 10A causes the determination result NG page indicating that it is not effective to apply the assistance device to the user to be displayed on the terminal device 20A (step S239). “S239: Determination result NG page” in FIG. 5 is an example of the determination result NG page. The user can confirm that it is not effective to apply the assistance device by referring to the determination result NG page displayed on the display 23 of the terminal device 20A.

On the other hand, in a case where the third input result is an answer indicating that the improvement of walking motion is demanded (when the button B1 (Yes) has been pressed), the first determiner 12 of the medical information providing device 10A transmits information on a measurement page that enables measurement of physical information to the terminal device 20A via the communicator 11 (step S227).

Then, the display 23 of the terminal device 20A displays the measurement page on the basis of the information on the measurement page received from the medical information providing device 10A (step S229). “S229: Measurement page” in FIG. 5 is an example of the measurement page. The measurement page includes “Press the terminal device on your chest or tummy and walk about 5 m.” and a button B3 (Start) for starting measurement. The terminal device 20A performs measurement of the physical information on the basis of pressing of the button by the user (step S231). In a process of this measurement, the acceleration sensor 26 and the angular velocity sensor 27 provided in the terminal device 20A measure an acceleration and an angular velocity when the user walks with the terminal device 20A pressed against a trunk such as a tummy or a chest. Then, the communicator 21 of the terminal device 20A transmits a measurement result of the physical information to the medical information providing device 10A (step S233).

Then, the first determiner 12 of the medical information providing device 10A determines whether or not it is effective to apply the assistance device to the user on the basis of the measurement result of the physical information received from the terminal device 20A (step S235). For example, the first determiner 12 of the medical information providing device 10A determines whether or not it is effective to apply the assistance device to the user on the basis of, for example, a result of a determination as to whether or not there is periodicity in the walking motion indicated by the measurement result or whether or not the bilateral symmetry of the walking motion deteriorates. In this case, the first determiner 12 of the medical information providing device 10A may determine the type of assistance device suitable for the user. The first determiner 12 of the medical information providing device 10A transmits information on a determination result page indicating the determination result to the terminal device 20A via the communicator 11 (step S237).

Then, the display 23 of the terminal device 20A displays a determination result page on the basis of the information on the determination result page received from the medical information providing device 10A (step S239). “S239: Determination result OK page” and “S239: Determination result NG page” in FIG. 5 are examples of the determination result page. The user can confirm whether or not it is effective to apply the assistance device by referring to the determination result page displayed on the display 23 of the terminal device 20A. Thus, the process of the sequence diagram ends.

According to the second embodiment described above, it is possible to provide information on the assistance device suitable for the target. Although the example in which the first to third input pages are divided and displayed on the terminal device 20A has been described in the second embodiment, a plurality of questions may be displayed on one input page and answers to the plurality of questions may be input at a time. The first determiner 12 of the medical information providing device 10A may determine that it is effective to apply the assistance device in a case where an answer indicating that rehabilitation is being performed has been obtained from the user.

### Third Embodiment

Hereinafter, a third embodiment will be described. A medical information providing device 10B included in a medical information providing system of a third embodiment is different from that of the first embodiment in that the medical information providing device 10B determines whether or not it is effective to apply the assistance device to the target using a determination model 17C generated by machine learning. Therefore, for a configuration or the like, the drawings and related description described in the first embodiment are used, and detailed description thereof will be omitted.

### [Configuration of Medical Information Providing Device 10B]

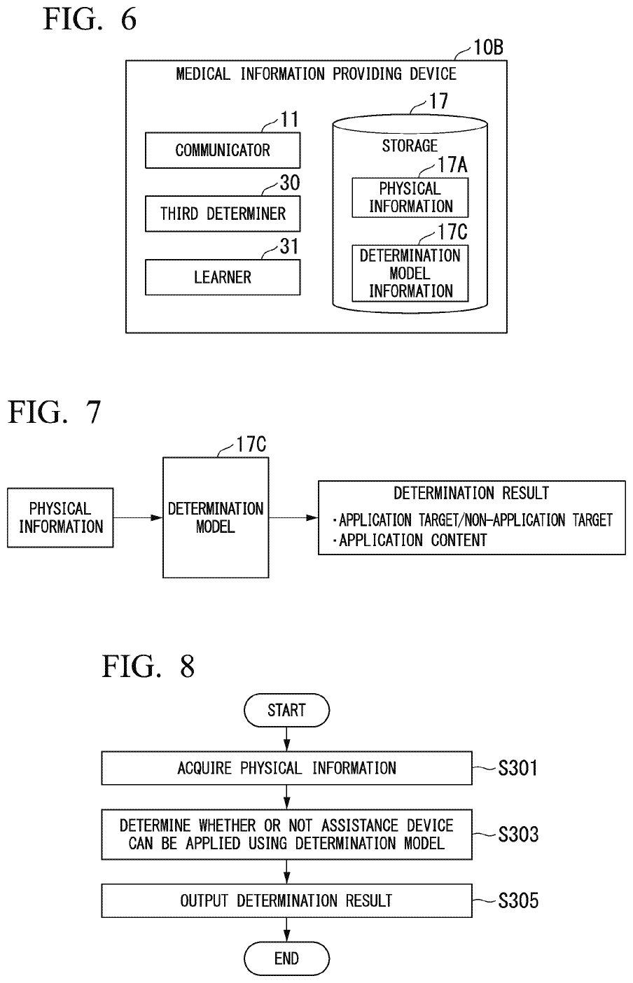

FIG. 6 is a functional block diagram of the medical information providing device 10B of the third embodiment. The medical information providing device 10B further includes, for example, a third determiner 30 and a learner 31, in addition to the communicator 11 and the storage 17 in the first embodiment. The third determiner 30 determines whether or not it is effective to apply the assistance device to the target using the determination model 17C stored in the storage 17.

FIG. 7 is a diagram showing a configuration of the determination model 17C of the third embodiment. The determination model 17C is a model learned to output application information of an assistance device to a target when physical information is input. The learner 31 generates the determination model 17C by learning a relationship between the physical information and the application information for the assistance device to the target.

The learner 31 learns, as learning data, the physical information of the user who has used the assistance device in the past and a result of using the assistance device (progress information indicating whether or not the use has been effective). For example, the progress information may be acquired by providing an input page for receiving an input of the progress information to the terminal device 20 and allowing the user to input the progress information. Alternatively, the progress information may be acquired by providing a function of monitoring progress information of the target (for example, a degree of improvement in the bilateral symmetry of the walking motion) to the assistance device, and the assistance device periodically uploading a monitoring result as degree-of-improvement data to a server such as the medical information providing device 10B. The determination model 17C is realized by, for example, a deep neural network (DNN). The determination model 17C may be realized by a convolutional neural network (CNN). The determination model 17C may define a determination line for clustering physical information in a nonlinear classifier such as support vector machine (SVM).

### [Processing Flow of Medical Information Providing Device 10B]

Next, a processing flow of the medical information providing device 10B will be described. FIG. 8 is a sequence diagram showing an example of a process of the medical information providing device 10B according to the third embodiment. First, the communicator 11 of the medical information providing device 10B acquires physical information of a target transmitted by the terminal device 20 (step S301).

Then, the third determiner 30 of the medical information providing device 10B inputs the acquired physical information of the target to the determination model 17C stored in the storage 17, and acquires application information for the assistance device that is output by the determination model 17C to determine whether or not it is effective to apply the assistance device to the target (step S303).

Then, the third determiner 30 of the medical information providing device 10B inputs the application information for the assistance device to the target, to the terminal device 20 via the communicator 11 (step S305). Thus, the process of the flowchart ends.

According to the third embodiment described above, it is possible to provide information on the assistance device suitable for the target. By using the model learned to output the application information for the assistance device when the physical information is input, it is possible to use a variety of pieces of physical information as a material for a determination as to whether or not an assistance device can be applied, and to provide information on the assistance device suitable for the target.

The example in which it is determined whether or not the assistance device can be applied in the medical field has been described in the above embodiment, but the present invention is not limited thereto and the present invention can also be applied to improvement of predetermined operations such as a form in the sports field. For example, in the medical information providing device 10, information on a form of the user may be acquired as the physical information, a habit (trend) of the form may be analyzed, and an operation of the assistance device may be controlled so that a difference between the acquired form and an ideal optimal form is reduced. For example, information on the form may be acquired by providing a function of monitoring the form of the user to the assistance device and the assistance device uploading a result of monitoring to a server such as the medical information providing device 10. A machine learning scheme may be adopted, a model using an optimal form as training data may be prepared, and the operation of the assistance device may be controlled using the model.

The example in which the medical information providing device 10 and the terminal device 20 are configured as separate devices has been described in the above embodiment, but the present invention is not limited thereto and the medical information providing device 10 and the terminal device 20 may be configured as one device. For example, in a case where a function of the medical information providing device 10 and a function of the terminal device 20 are realized by one mobile phone such as a smartphone, these functions may be executed by an application program installed in the smartphone.

An aspect for carrying out the present invention has been described above using the embodiments, but the present invention is not limited to the embodiments at all, and various modifications and substitutions may be made without departing from the gist of the present invention.

What is claimed is:

1. A computer-readable non-transitory storage medium storing a program, the program causing a computer to:
acquire physical information of a user; and
determine whether or not it is effective to apply an assistance device assisting in a personal motion to the user on the basis of the acquired physical information.

2. The storage medium according to claim 1, wherein the program is configured to cause the computer to determine whether or not it is effective to apply the assistance device to the user in a case where the physical information includes information on skeletal movement trouble of the user.

3. The storage medium according to claim 1,
wherein the assistance device is a walking assistance device, and
the program is configured to cause the computer to determine that it is effective to apply the assistance device to the user in a case where there is periodicity in a walking motion of the user indicated by the physical information.

4. The storage medium according to claim 1,
wherein the assistance device is a walking assistance device, and
the program is configured to cause the computer to determine that it is effective to apply the assistance device to the user in a case where bilateral symmetry of a walking motion of the user indicated by the physical information deteriorates.

5. The storage medium according to claim 1,
wherein the program is configured to cause the computer to determine a type of assistance device suitable for the user on the basis of the acquired physical information in a case where it has been determined that it is effective to apply the assistance device to the user.

6. The storage medium according to claim 1,
wherein the program is configured to cause the computer to:
further acquire a result which is an answer of the user to a question about the physical information; and
determine whether or not it is effective to apply the assistance device to the user on the basis of both the acquired answer result and the acquired physical information.

7. The storage medium according to claim 1,
wherein the program is configured to cause the computer to generate a page on which an answer to a question about the physical information is able to be input and a page on which measurement data regarding a body is able to be acquired.

8. The storage medium according to claim 1,
wherein the program is configured to cause the computer to determine whether or not it is effective to apply the assistance device to the user, using a model learned to output application information for the assistance device when the physical information is input.

9. An information providing method comprising:
acquiring, by a computer, physical information of a user; and
determining, by the computer, whether or not it is effective to apply an assistance device assisting in a personal motion to the user on the basis of the acquired physical information.

10. An information providing device comprising:
an acquirer configured to acquire physical information of a user; and
a first determiner configured to determine whether or not it is effective to apply an assistance device assisting in a personal motion to the user on the basis of the physical information acquired by the acquirer.
